(19) United States
(12) Patent Application Publication
(10) Pub. No.: US 2007/0163585 A1
(43) Pub. Date: Jul. 19, 2007

(54) Method for accessing abdominal cavity and medical procedure via natural orifice

(75) Inventors: Takefumi Uesugi, Tokyo (JP); Kenji Noda, Tokyo (JP); Takayasu Mikkaichi, Tokyo (JP); Kunihide Kaji, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., TOKYO (JP)

(21) Appl. No.: 11/331,974

(22) Filed: Jan. 13, 2006

(51) Int. Cl. A61M 16/00

(52) U.S. Cl. 128/204.18

(57) ABSTRACT

The method for accessing the abdominal cavity according to the present invention, includes: introducing a first flow path into the abdominal cavity; introducing a second flow path into the hollow organ from the natural orifice of the living body, and performing a pressure control, using the first flow path and the second flow path, so that the pressure within the hollow organ is lower than the pressure of the abdominal cavity, and forming an opening in the wall of the hollow organ from the inside of the hollow organ when the pressure within the abdominal cavity, as accomplished by pressure control, is equal to or lower than the pressure of the abdominal cavity, and inserting a device for performing a medical procedure through the opening.

### BACKGROUND OF THE INVENTION

1. Field of the Invention

The present invention relates to a method for accessing the abdominal cavity and a medical procedure via a natural orifice.

2. Description of the Related Art

In the case of accomplishing medical intervention (including observation and treatment and the like; as applied hereafter) to the viscera of the human body, in lieu of cutting deeply into the abdominal wall, surgery in which multiple openings are cut into the abdominal wall, through which are respectively inserted such medical devices as rigid laparoscopes, and forceps and the like, is well-known. Since this is accomplished with only small openings, there is the advantage of minimal invasion and the rapid recovery of the patient.

In recent years, as a method of further reducing the invasion of the patient, it has been proposed that operations be accomplished by inserting an endoscope through such natural orifices as the patient's mouth, a nostril, or anus. Examples of such medical activity are disclosed in U.S. Pat. No. 5,458,131. By inserting a flexible endoscope from the mouth of a patient in which insufflation of the abdomen has been initiated, the endoscope can then be sent to the abdominal cavity through an opening formed in the stomach wall, and used to monitor the inside of the abdominal cavity. Furthermore, organs can be treated using treatment devices passed through the endoscope, or through other openings to the stomach, or from the opening in the Sigmoid colon formed from the anus, and upon completion of the operation within the abdominal cavity, the treatment devices can be removed and the openings closed. At the time of closing the openings, the tissue can be aspirated by binding the periphery of the opening, and constraining it with an O ring.

### SUMMARY OF THE INVENTION

The object of the present invention relates to performing a medical procedure for approaching the inside of the abdominal cavity by forming an opening in the wall of a hollow organ from the natural orifice of the body, and provides a method for accomplishing a medical procedure by appropriately controlling the pressure within the abdominal cavity and hollow organs.

A method for accessing the abdominal cavity according to a first aspect of the present invention comprises method for accessing an abdominal cavity, comprises: introducing a first flow path into the abdominal cavity; introducing a second flow path into a hollow organ from a natural orifice of the living body; performing a pressure control so that a pressure within the hollow organ is lower than a pressure of the abdominal cavity by using of the first flow path and the second flow path; forming an opening in a wall of the hollow organ from the inside of the hollow organ when the pressure within the hollow organ is equal to or less than the pressure within the abdominal cavity, in the step of performing a pressure control; and inserting a device for performing a medical procedure through the opening.

A medical procedure accomplished through a natural orifice according to a second aspect of the present invention comprises introducing a first flow path into an abdominal cavity; introducing the second flow path into the hollow organ from the natural orifice of the living body; performing a pressure control using the first flow path and the second flow path so that a pressure within the hollow organ is equal to or less than a pressure of the abdominal cavity; and in a state in which, by performing a pressure control, the pressure within the hollow organ is made equal to or less than the pressure within the abdominal cavity, withdrawing the device which is inserted from the natural orifice of the living body and is introduced into the abdominal cavity through an opening formed in the hollow organ, from the abdominal cavity.

### BRIEF DESCRIPTION OF THE DRAWINGS

FIG. 1A is a diagram showing the entire structure of a medical system which includes an example of a device for accomplishing a medical procedure.

FIG. 1B is a partially enlarged diagram of FIG. 1A.

FIG. 2 is a diagram showing the structure of an air supply device.

FIG. 3 is a flow chart of the pressure regulation.

FIG. 4 is a diagram illustrating a medical procedure, and a diagram which forms an opening in the stomach.

FIG. 5 is a diagram which shows one mode of performing a medical procedure in which an endoscope is introduced to the abdominal cavity through an opening in the stomach.

FIG. 6A is a diagram illustrating the air supply route according to another embodiment.

FIG. 6B is an enlarged diagram of the stopper shown in FIG. 6A.

FIG. 6C is an enlarged diagram of the distal end of the endoscope shown in FIG. 6A.

FIG. 6D is an enlarged diagram of a local injection needle.

FIG. 7 is a diagram in which the local injection needle through the endoscope is inserted to the stomach wall, insufflation of the abdominal cavity.

FIG. 8 is a diagram showing the structure of an air supply device.

FIG. 9A and is a diagram illustrating the air supply route according to another embodiment.

FIG. 9B is an enlarged diagram of the overtube shown in FIG. 9A.

FIG. 10 is a flowchart of the pressure regulation.

FIG. 11 is a diagram showing the structure an air supply device.

FIG. 12 is a diagram in which the local injection needle through the endoscope is inserted to the stomach wall, insufflation of the abdominal cavity.

FIG. 13 is a diagram showing the overtube pressing into the stomach.

FIG. 14 is a flowchart of the pressure regulation.

FIG. 15 is a diagram in which the stomach wall is aspirated into the overtube.

FIG. 16 is a diagram showing the formation of an opening using a high frequency knife on the aspirated stomach wall.

FIG. 17 is a diagram illustrating the air supply route according to another embodiment.

FIG. 18 is a cross-sectional diagram showing the structure of an end cover.

FIG. 19 is a diagram of the stomach wall being aspirated into the overtube.

FIG. 20 is a diagram showing the formation of an opening of the aspirated stomach wall by means of a high frequency knife.

FIG. 21 is a diagram showing an embodiment in which a tube used for air expulsion is separately inserted into the endoscope.

FIG. 22 is a diagram showing an embodiment in which there is an attached tube used for air exhaust delivery on the external side of the endoscope.

FIG. 23 is a diagram showing an embodiment in which there is an attached tube used for air exhaust on the outer side of the overtube.

FIG. 25 is a cross-sectional diagram along the line XXV-XXV of FIG. 24.

### DETAILED DESCRIPTION OF THE PREFERRED EMBODIMENTS

A detailed explanation is provided hereafter of the preferred embodiment. Moreover, hereafter, the same labels will be applied to the same structural elements. in addition, the explanation of recovery will be abbreviated.

### First Embodiment

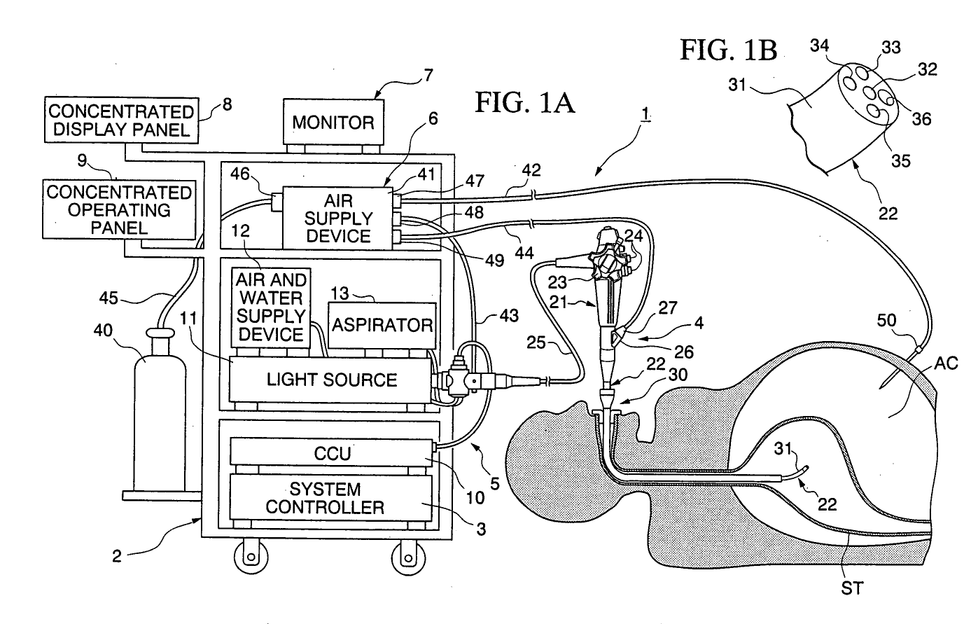

In FIG. 1A, a description is provided of the medical system used in the preferred embodiment. Medical system 1 includes a system controller 3 mounted on a cart 2, an endoscope system 5 which includes an endoscope 4 which is inserted into a body, an air supply system 6, a monitor 7 which is the display device, a concentrated panel 8, and a concentrated operating panel 9.

The system controller 3 entirely controls the medical system 1. To system controller 3, is connected a concentrated display panel 8, a concentrated operating panel 1, and an endoscope system 5 and the like through a communication line which is not shown, in order to accomplish bi-directional communication.

The endoscope system 5 accomplishes a medical procedures to a hollow organ or the abdominal cavity from the mouth of the patient, and includes the flexible the endoscope 4, a camera control unit (hereinafter referred to as CCU) 10, a light source 11, an air and water supply device 12, and an aspirator 13.

The endoscope 4 includes an operating unit 21 operated by an operator, and a long flexible insertion device is extendingly attached from the operating unit 21. The operating unit 21 is arranged with an angle knob 23 which bends the insertion device 22, and various buttons 24, connected to the system light source 11 by means of a universal cable 25. To the side of the operating unit 21 is attached a treatment tool insertion portion 26 for inserting the treatment tool, and to which a stopper 27 is tightly fitted. Moreover, in FIG. 1A, the endoscope 4 is inserted through the stomach ST through an overtube 30 which guides its insertion to within the body. However, the overtube is not necessarily required.

To the distal end of insertion unit 22 is attached a bendable end unit 31. As shown in FIG. 1B, on the distal end surface of the insertion unit 22 is arranged an observation device 32, an illumination device 33, an end opening of an air and water channel 34, an end opening of an aspiration channel 35, and an end opening of a the operation channel 36.

The observation device includes an observation optical system and photographic image device, which converts an in vivo optical image to an electrical signal, and outputs it to the CCU 10 through the universal cable 25. The CCU 10 converts an electrical signal sent from the observation device 32 to a video signal, and displays the in vivo optical image on a monitor 7 or the concentrated display panel 8. The illumination device 33 includes an illumination window and light guide, which provides in vivo illumination with illumination light supplied from the light source 11.

Air and water are supplied to the air and water supply channel 34 from the air and water supply device 12 through the universal cable 25. The aspiration channel 35 is connected to the aspirator 13 through the universal cable 25. An operation channel 36 is connected to the treatment tool insertion portion 26 on the side of the operating unit 21 passing within insertion unit 22. However, the structure of the endoscope 4 is not limited to this. For example, the aspiration channel 35 may be abbreviated, and aspiration may be accomplished through the use of the operation channel 36.

The monitor 7 receives a video signal output from the CCU 10, and the endoscope picture image is displayed. The display screen of the liquid crystal display is attached to the concentrated display panel 8. The concentrated display panel 9 outputs the endoscope video image to the display screen, at the same time as which the operating state of each device obtained from the system controller 3 is concentratedly displayed. A construction is such that, on the concentrated operation panel 9, input can be accomplished of various operations or settings using a touch sensor. Each system can be remotely operated using the concentrated operation panel 9.

The air supply system 6 has, as its primary structural elements, a gas cylinder 40 which is the supply source of the liquid used for insufflation, an air supply device 41, and the tubes 42, 43, and 44 which extend toward the patient from the air supply device 41. A gas cylinder 40 is filled, for example, with liquid carbon dioxide which is the high-pressure gas. A tube 45 used for the high-pressure gas extending from the gas cylinder 40 is coupled to a high-pressure metal cap 46 of the air supply device 6. Other than the high-pressure metal cap 46, to the air supply device 41 is attached an insufflation metal cap 47, a luminal metal cap 48, and a pressure measurement metal cap 49.

The tube 42 connected to the insufflation metal cap 47 is connected to an insufflation needle 50. The tube 43 connected to the luminal metal cap 48 is connected to the air and water supply channel 34 through the universal cable 25 of the endoscope 4. The tube 44 connected to the pressure measurement metal cap 49 is inserted from stopper 27 of the endoscope 4, and is led to the distal end of the operating channel 36. Each tube 42-44 is formed from silicon resin or Teflon (registered trademark).

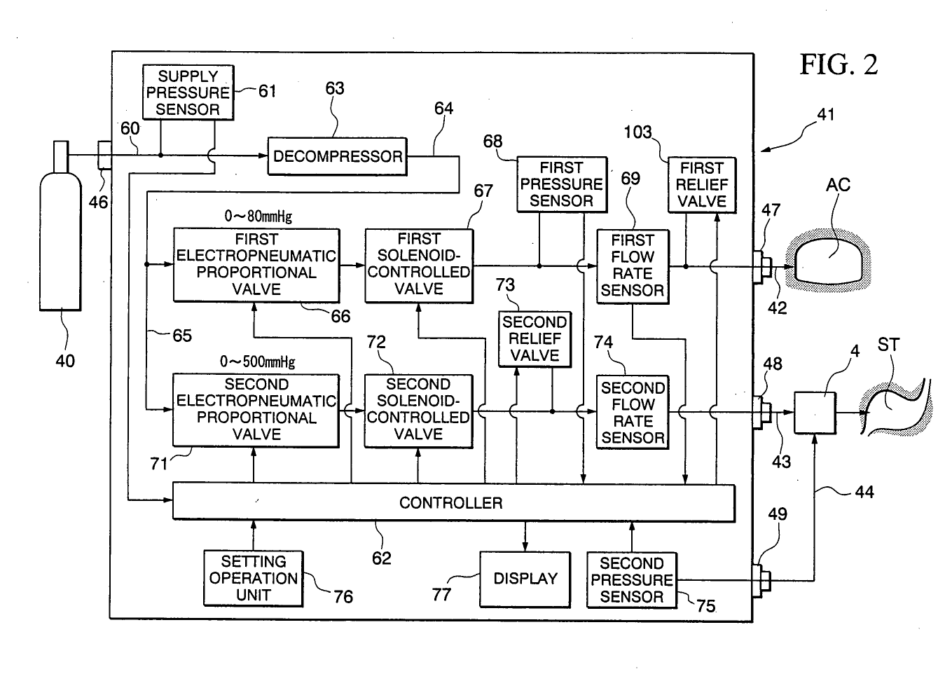

A summarized description is provided hereafter of the structure of the air supply device 41 shown in FIG. 2.

The air supply device 41 has a supply pressure sensor 61 attached in the flow path 60 connected to the high-pressure metal cap 46. The pressure of carbon dioxide gas supplied from the gas cylinder 40 is measured, and output to the controller 62. A decompressor 63 is attached downstream from the supply pressure sensor 61. The decompressor 63 reduces the specified pressure of the high-pressure gas. Downstream of the decompressor 63 is formed a first flow path 64 toward the insufflation metal cap 47, at the same time as which a second flow path 65, which branches from the first flow path 64, is extended toward the luminal metal cap 48.

In the first flow path 64 are attached, in order from the upstream side, a first electropneumatic proportional valve 66, a first solenoid-controlled valve 67, a first pressure sensor 68, a first flow rate sensor 69, and a first relief valve 103. The first electropneumatic proportional valve 66 changes the force of the decompression spring operating on the valve based on a control signal from the controller 62, electrically regulating the pressure of carbon dioxide gas. The first electropneumatic proportional valve 66 regulates the air supply pressure of the carbon dioxide gas within a range between 0 to 80 mmHg. The first pressure sensor 68 measures the pressure value Pa within the abdominal cavity AC through the first flow path 64. The first flow rate sensor 69 measures the flow rate of carbon dioxide gas flowing through the first flow path 64, and outputs it to the controller 62. The first relief valve 103 is a solenoid-controlled valve which accomplishes an open/close operation by means of a signal from the controller 62.

In the second flow path 65 is attached, in order from the upstream side, a second electropneumatic proportional valve 71, a second solenoid-controlled valve 72, a second relief valve 73, and a second flow rate sensor 74. The second electropneumatic proportional valve 71 regulates the air supply pressure of the carbon dioxide gas within a range between 0 to 500 mmHg by means of the control signal of the controller 62. The second relief valve 73 is a solenoid-controlled valve which accomplishes an open/close operation based on the control signal from the controller 62. The second flow rate sensor 74 measures the flow rate of carbon dioxide gas flowing through the second flow path 65, and outputs it to the controller 62.

Furthermore, to the pressure measurement metal cap 49 is attached a second pressure sensor 75. The second pressure sensor 75 measures the pressure value Ps within the stomach ST, and its output is input to the controller 62. Moreover, a setting operation unit 76 and a display 77 are also connected to the controller 62. The setting operation unit 76 and the display 77 receive a particular display or operation related to the air supply device 41, and are provided, for example, on the front surface panel of the air supply device 41. As the setting operation unit 76, an electric power switch, an air supply start button, or an air supply suspension button may be used. As the display 77, use can be made of such as the residual gas amount display which accomplishes display based on the output of the supply pressure sensor 61.

An explanation is provided next concerning operations used when performing medical procedures using the medical treatment system 1 shown in FIG. 1.

An explanation is provided hereafter of the treatment of organs or tissues (hereinafter referred to as the target sites) which are the target of a specific medical procedure, in which the endoscope 4 is inserted from the patient's mouth, which serves as the natural orifice of the body. However, the natural orifice for inserting the endoscope 4 is not limited to the use of the mouth, and use may also be made of a nostril or the anus. In addition, as medical treatment, various procedures may be applied, including those relating to the application of sutures, making observations, incision, and cell collection.

The insertion unit 22 of the endoscope 4 is inserted from the mouth of the patient, and the distal end of the insertion unit 22 is guided to within the stomach ST. In addition, the insufflation needle 50 is inserted into the abdominal cavity AC through the abdominal wall of the patient.

Initially, air is delivered into the stomach ST through a tube 43 from the air supply device 41, inflating the stomach ST, thereby enabling visual inspection of an incision target site and by means of the endoscope 4. At this time, an operator accomplishes the operation of a condensed operation panel 9 or the air supply device 41 shown in FIG. 1 to select the second pipe channel 65, as well as setting pressure within the stomach ST. The controller 62 shown in FIG. 2 operates the second electropneumatic proportional valve 71, thereby opening the second solenoid-controlled valve 72. The degree of opening of the second electropneumatic proportional valve 71 is set by the controller 62, based on the output of a second flow rate sensor 74. Carbon dioxide gas within the air cylinder 40 is delivered to the air and water supply channel 34 of the endoscope 4 through the tube 43 from the second of flow path 65, which is delivered to the inside of the stomach ST from the distal end opening. The pressure within the stomach ST is increased with the flow of carbon dioxide gas.

The pressure value Ps within the stomach ST is detected by the second pressure sensor 75 through the tube inserted into the operating channel 36. The controller 62 compares the pressure value Ps (actually measured value) of the second pressure sensor 75 with the target pressure set by the operator. If the pressure value Ps does not reach the target pressure, then the degree of opening of the second electropneumatic proportional valve 71 is adjusted according to the difference between the target pressure and the pressure value Ps, thereby changing the air supply pressure. On the other hand, if the pressure value Ps is greater than the target pressure, the second solenoid-controlled valve 72 is closed, cutting off the supply of carbon dioxide gas to the stomach ST, and atmospheric release is accomplished by opening the second relief valve 73, if necessary. In this manner, the air supply device 41 accomplishes control so as to maintain the pressure within the stomach ST at a specific level.

Confirmation of the position for cutting the stomach ST is accomplished by the observation device 32 of the endoscope 4. At this time, a high frequency treatment tool or a clip or the like, may also be used by making a mark in the incision target position the body.

When driving the air supply system 6, performing the insulation of the abdominal cavity AC, and cutting the stomach wall, care must be taken to assure that other organs are not injured. At this time, the operator operates the concentrated operation panel 9 or air supply unit 41, selecting so as to deliver air to the tube 42, thereby establishing internal pressure within the abdominal cavity AC. The controller 62 shown in FIG. 2 operates the first electropneumatic proportional valve 66, opening the first solenoid-controlled valve 67. The degree of opening of the first electropneumatic proportional valve 66 is set by the controller 62 based on the output of the first pressure sensor 68 and the first flow rate sensor. Carbon dioxide gas within the compressed gas container 40 is sent to the abdominal cavity from the insufflation needle 50 through tube 42 from the first flow path 64. The pressure within the abdominal cavity AC is increased with the inflow of carbon dioxide gas, with the pressure value Pa of the abdominal cavity AC being detected by the first pressure sensor 68. When measuring the pressure within the abdominal cavity AC, the controller 62 closes the first solenoid-controlled valve 67. Since the supply of carbon dioxide gas from the air cylinder 40 is suspended, the measurement value of the first pressure sensor 68 after the lapse of a predetermined time period becomes the pressure value Pa within the abdominal cavity AC.

The controller 62 compares the pressure value Pa (actual measured value) of the abdominal cavity AC measured by the first pressure sensor 68 with a target pressure set by the operator. If the pressure value Pa does not reach the target pressure, the degree of opening of the first electropneumatic proportional valve 66 is adjusted according to the difference between the target pressure and the pressure value Pa, thereby changing the air supply pressure. On the other hand, if the pressure value Pa is greater than the target pressure, the first solenoid-controlled valve 67 is closed, and the supply of carbon dioxide gas to the abdominal cavity AC is suspended. Subsequently, opening the first relief valve 103 and release to the atmospheric pressure is accomplished, then the pressure value Pa within the abdominal cavity AC is decreased. In this manner, the air supply device 41 accomplishes control to maintain the pressure within the abdominal cavity AC at a specified pressure.

Moreover, if the insufflation of the abdominal cavity AC is first performed, it is suffice that the pressure within the stomach ST is made higher than the pressure within the abdominal cavity AC in order to inflate the stomach ST.

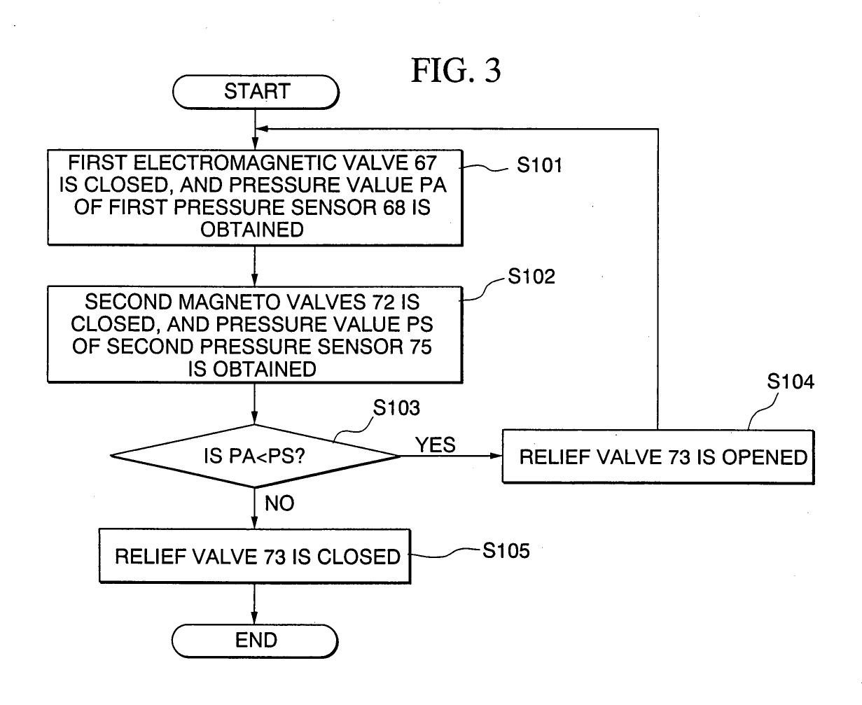

Once the target site for incising the body has been confirmed, the air within the stomach ST is evacuated by the air supply device 41 and the pressure value Ps within the stomach is reduced to the pressure value Pa of the abdominal cavity AC or less. As shown in FIG. 3, the first solenoid-controlled valve 67 is closed, and the pressure value Pa of the first pressure sensor 68 is obtained (in step S101). Next, the second solenoid-controlled valves 72 is closed, and the pressure value Ps of the second pressure sensor 75 is obtained (in step S102). The controller 62 compares the values of the pressure values Pa and Ps, and if the pressure value Ps within the stomach ST is greater than the pressure value Pa of the abdominal cavity (YES in step S103), then a second relief valve 73 is opened (in step S104). Since the second relief valve 73 is provided to the second flow path 65 of the air supply device 41, carbon dioxide gas within the stomach ST is discharged outside the body through the air and water supply channel 34. Subsequently, returning to step S101, steps S101-S104 are repeated until the pressure value Ps within the stomach ST reaches the pressure value Pa of the abdominal cavity AC or less. During this time, since the second relief valve 73 is in the open state, the pressure within the stomach ST is gradually reduced. If the pressure value Ps within the stomach ST falls to the pressure value Pa of the abdominal cavity AC or less (No in step S103), the second relief valve 73 is closed (in step S105), at which point the processing is terminated. Moreover, if the pressure value Ps within the stomach ST becomes a low specified value which is lower than the pressure value Pa, then advancement may be made to step S105.

After adjusting the pressure of the stomach ST, a treatment tool used for making incision, e.g., a high frequency knife, is passed through operation channel 36 of the endoscope 4, and the stomach wall is cut. If the high-frequency knife is used for marking, incision can also be made with the knife.

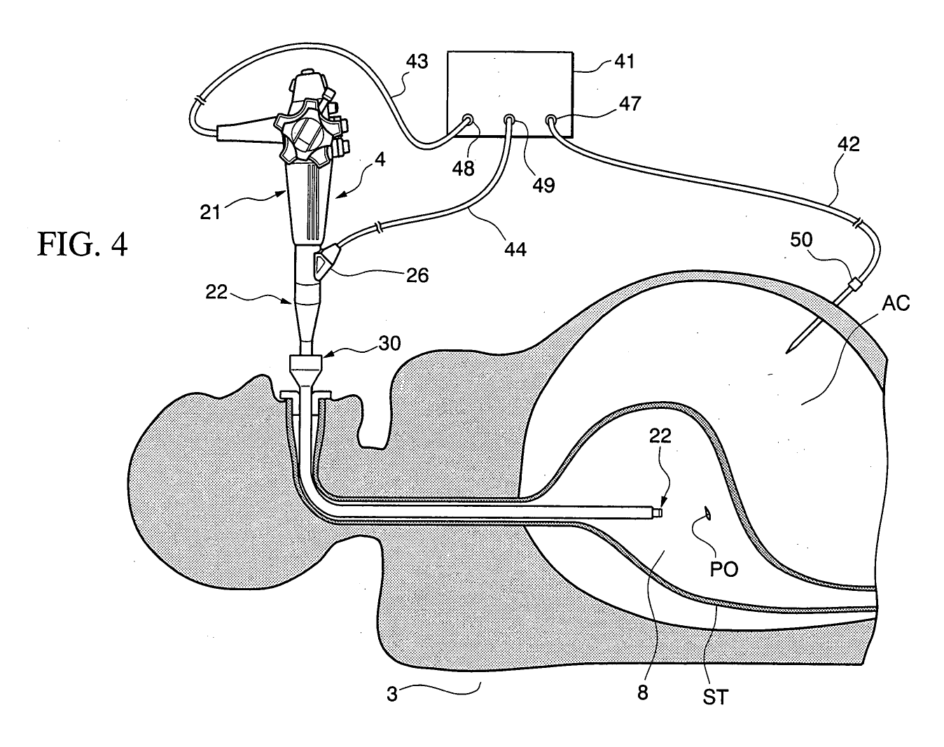

As shown in FIG. 4, an opening PO is formed in the stomach wall. However, since the pressure value Ps within the stomach ST is equal to or less than the pressure value Pa within the abdominal cavity AC, outflow of carbon dioxide gas or any other fluid (hereinafter referred to as a fluid) from the stomach ST to the abdominal cavity AC can be prevented, keeping the abdominal cavity AC clean, and preventing infection, from the viewpoint of using pressure control within the stomach ST and the abdominal cavity AC.

The insertion unit 22 is advanced, and the abdominal cavity AC is accessed by passing the endoscope 4 and the overtube 30 through the opening PO made by cutting the stomach wall. At this time, carbon dioxide gas is delivered from the first flow path 64 of the air supply device 41, and insufflation of the abdominal cavity is performed. While performing medical procedures within the abdominal cavity AC, the second solenoid-controlled valve 72 is maintained in its closed state, and no air is delivered from the second flow path 65. In this step, the insufflation is performed again in order to define a space for performing the medical procedure within the abdominal cavity AC.

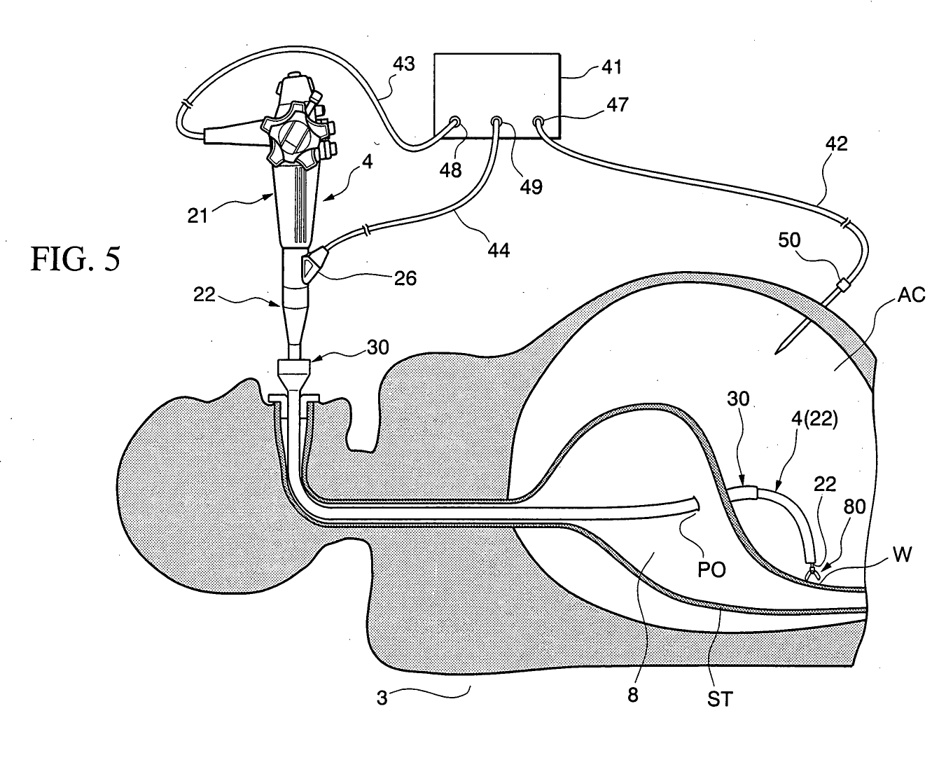

As shown in FIG. 5, the endoscope 4 is advanced in the abdominal cavity AC in which insufflation has been performed, and the distal end surface of the endoscope 4 is faced toward the target site W. While observing the target site W with the observation device 32, treatment is accomplished using the treatment tool 80 passed through the operating channel 36. For example, forceps are used to remove tissue by passing it through the operating channel. In addition, a high frequency treatment tool is able to burn a target site W by passing it through the operating channel 36. Preferably, smoke produced at the time of burning is discharged to the outside through the aspiration channel 35 or the air supply device 41, ensuring the clear field of view of the endoscope 4.

Upon completion of the medical procedure, the endoscope 4 and the overtube 30 are withdrawn to the stomach ST. Since no carbon dioxide gas is supplied to the stomach ST since cutting the stomach wall until the completion of the medical procedure, the pressure value Ps within the stomach STs is equal to or less than the pressure value Pa of the abdominal cavity AC. Furthermore, since the opening PO of the stomach ST is pressed and broadened by the overtube 30, the opening PO is spontaneously closed by removing the overtube 30. Accordingly, even if the endoscope 4 and the overtube 30 are withdrawn to the stomach ST, there is no outflow of fluid from the stomach ST to the abdominal cavity AC.

In this instance, an operator, prior to withdrawing the endoscope 4 and the overtube 32 to within the stomach ST, may confirm if the pressure value Ps within the stomach ST is equal to or less than the pressure value Pa of the abdominal cavity AC. At this time, in order to detect the pressure value within the stomach ST, a separate pipe channel is inserted into the stomach ST from the mouth (natural orifice) of the patient, and the pressure value Ps is confirmed by viewing a pressure gauge connected to the pipe channel. By so doing, the outflow of fluid from the stomach ST to the abdominal cavity AC can be further reliably prevented. Moreover, if the pressure value Ps within the stomach ST is equal to or less than the pressure value Pa of the abdominal cavity AC, then pressure within the stomach ST is reduced in accordance with the flow shown in FIG. 3.

Subsequently, the opening PO is sutured from the inside of the stomach ST using a treatment tool for suturing by passing it through the operating channel of the endoscope 4, which has been returned to the stomach ST. In confirming that the opening PO has been completely sutured, a leak test should be accomplished. In the leak test, water is supplied to the stomach ST from the air and water supply channel 34 of the endoscope 4, into which the location of the suture is immersed. If the suture is incomplete, bubbles are produced within the stomach ST. If no bubbles are produced, the water within the stomach ST will be aspirated through the aspiration channel 35 of the endoscope 4. Suspending the supply of carbon dioxide gas to the abdominal cavity AC, the tube 42 is removed from the insufflation needle 50, and the gas is discharged into the atmosphere from within the abdominal cavity AC. In addition, the controller 62 makes the second relief valve 73 open to discharge carbon dioxide gas within the stomach ST, thereby returning the stomach ST to atmospheric pressure. Subsequently, the insufflation needle 50 is removed from the abdominal wall, and the endoscope 4 and the overtube 30 is removed from the patient's mouth.

As explained above, in this embodiment, when performing a medical procedure within the abdominal cavity AC, using an endoscope 4 inserted from the mouth of a patient, and prior to making an incision in the stomach ST, pressure control is accomplished so that the pressure value Ps within the stomach ST is equal to or less than the pressure value Pa of the abdominal cavity AC. Ordinarily, in order to confirm the opening of the stomach wall of a target site, the pressure within the stomach ST must be higher than the pressure within the abdominal cavity AC. In such a case, by releasing the pressure within the stomach ST, if necessary, outflow can be prevented of fluid from the stomach S to the abdominal cavity AC, when cutting the stomach ST, thereby preventing infection.

In addition, in this embodiment, during the period in which a medical procedure is performed in the abdominal cavity AC, since the pressure value Ps within the stomach ST is equal to or less than the pressure Pa of the abdominal cavity AC, after the completion of a medical procedure, when the endoscope 4 and the overtube 30 are withdrawn from the abdominal cavity AC to the stomach ST as well, outflow of fluid from the stomach ST to the abdominal cavity AC can be prevented. As in the case of the time of advancement, the abdominal cavity AC can be maintained in a clean state, preventing infection.

### Second Embodiment

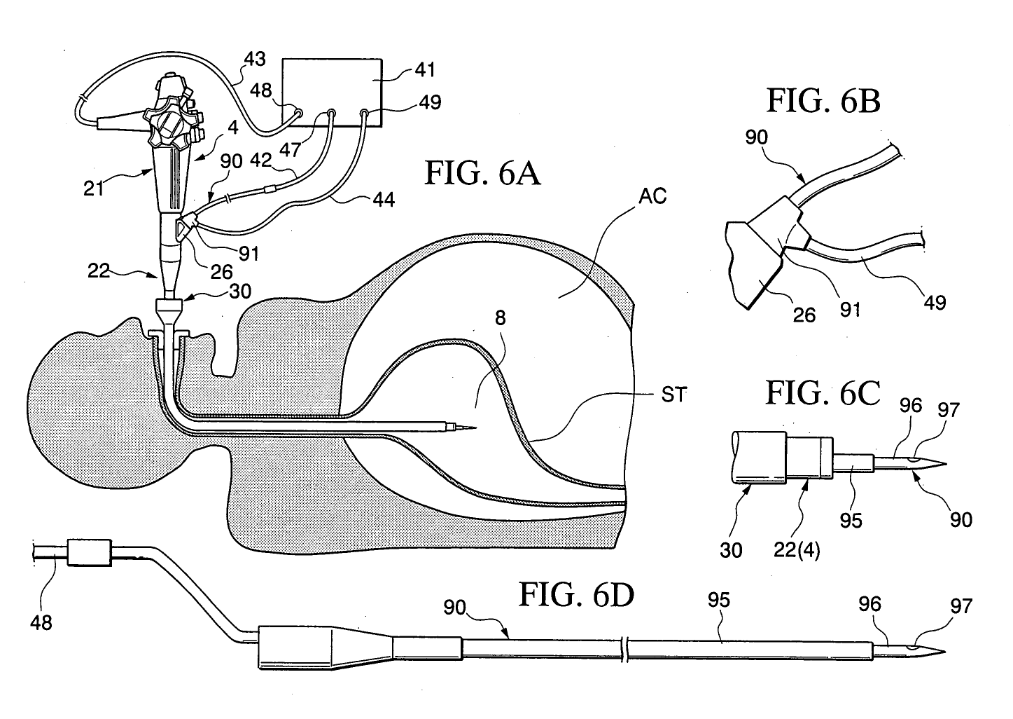

As shown in FIG. 6 A to FIG. 6 D, the luminal metal cap 48 of the air supply device 41 is connected to the air and water supply channel 34 of the endoscope 4 through the tube 43. The insufflation metal cap 47 is connected to the lumen of a local injection needle 90 which is the treatment tool through the tube 42. The local injection needle 90 is passed through the operating channel 36 via a stopper 91 attached to the treatment tool insertion portion 26 of the endoscope 4. The stopper 91 is branched into two, a tube 44 used for pressure measurement being inserted from the insertion opening of the branched portion.

The local injection needle 90 includes a needle member 96 that can be freely advanced to/withdrawn from a protective sheath 95. A lumen is formed within the needle member 96. The proximal end of the lumen is connected to a tube 42. The distal end of the lumen forms a side facing opening 97, in the vicinity of the sharp end of the needle member 96.

An explanation is provided hereafter of medical procedures used in the present embodiment, which differs from the first embodiment only in that it provides a route for supplying carbon dioxide gas at the time of insufflation.

After connection with the air supply device 41 through the local injection needle 90 and the operating channel 36 of the endoscope 4, the endoscope 4 is inserted into the patient's stomach ST.

Next, carbon dioxide gas is supplied to the air and water supply channel 34 to inflate the stomach ST. The pressure value Ps within the stomach ST is detected by a second pressure sensor 75 of the air supply device 41 inserted through the tube 44 from the two-way stopper 91.

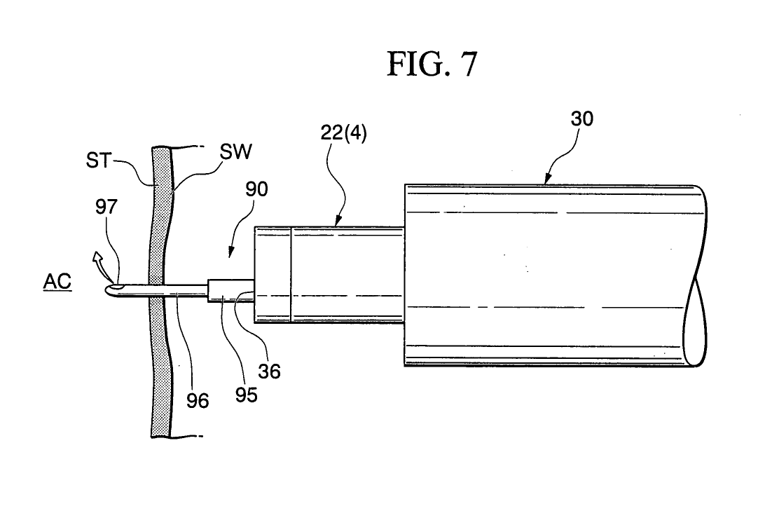

As shown in FIG. 7, a needle member 96 of the local injection needle 90 is advanced, penetrating the stomach wall SW. At this time, the local injection needle 90 is pressed into the stomach wall SW until the distal end opening 97 of the lumen is exposed to the abdominal cavity AC.

When the abdominal cavity AC is subjected to insufflation, carbon dioxide gas is supplied from the first flow path 64 of the air supply device 41, and is provided to the abdominal cavity AC through the lumen within the local injection needle 90. The pressure value Pa of the abdominal cavity AC is detected by the first pressure sensor 68 (see FIG. 2).

In confirming the target site for cutting the stomach ST, the pressure value Ps within the stomach ST is made to be higher than the pressure value Pa of the abdominal cavity AC.

When cutting the stomach wall SW, the second relief valve 73 (see FIG. 2) of the air supply device 41 releases to the atmospheric pressure, and accomplishes pressure control within the stomach ST. If the pressure value Ps within the stomach ST is equal to or less than the pressure value Pa of the abdominal cavity AC, an opening PO is formed by cutting the stomach wall SW. When inserting the endoscope 4 into the abdominal cavity AC through the opening PO, the local injection needle 90 is withdrawn from the stomach wall SW. After cutting the stomach wall SW, the insertion unit 22 is advanced to access the abdominal cavity AC by passing the endoscope 4 and the overtube 30 through the thus formed opening PO. At this time, carbon dioxide gas is supplied from a second flow path 65 of the air supply device 41, and after performing insufflation of the abdominal cavity AC, a medical procedure is executed, during which the first solenoid-controlled valve 67 remains in the closed state, and air supply is not accomplished from the first flow path 64. The procedures thereafter are the same as those of the first embodiment.

In this embodiment, the outflow of fluid from the stomach ST to the abdominal cavity AC can be prevented by controlling the pressure within the stomach ST and the pressure within the abdominal cavity AC, which can ordinarily be maintained in the clean state, thereby preventing infection. Furthermore, since there is no need for inserting the insufflation needle 50 in the abdominal wall, the medical procedures can be accomplished without causing an injury to the patient.

### Third Embodiment

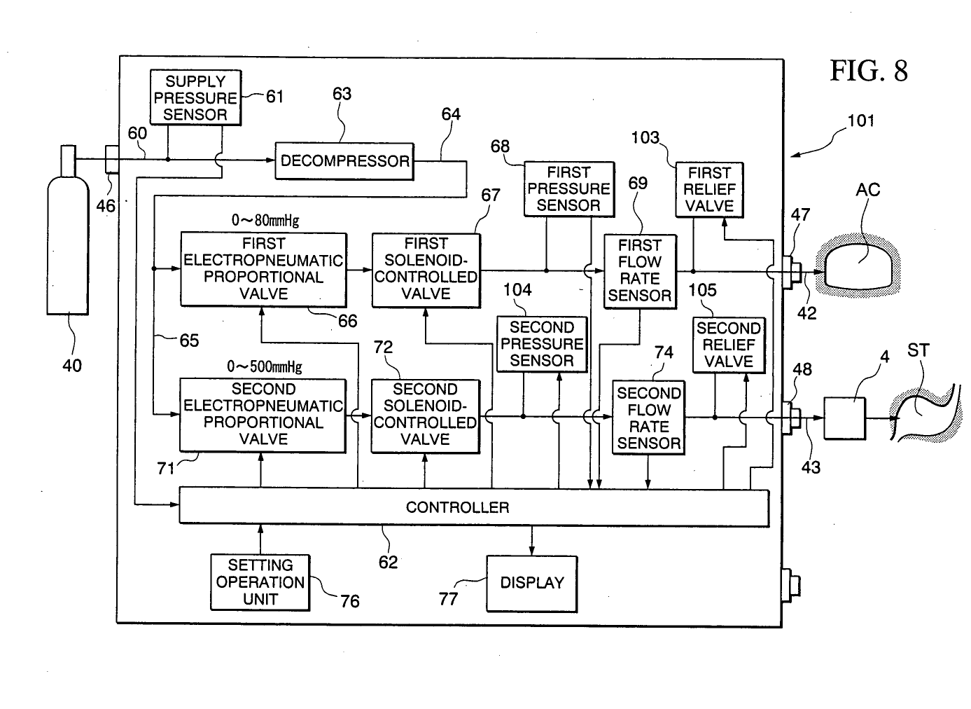

The structure of the air supply device used in this embodiment is shown in FIG. 8.

An air supply device 101 is provided with the second solenoid-controlled valve 72 in the second flow path 65 branch from the decompressor 63, a second pressure sensor 104, the second flow rate sensor 74, and a second relief valve 105 in this order, and is connected to the tube 43. The first and second relief valves 103 and 105 respectively are solenoid-controlled valves capable of being atmospherically released by the control signal of the controller 62.

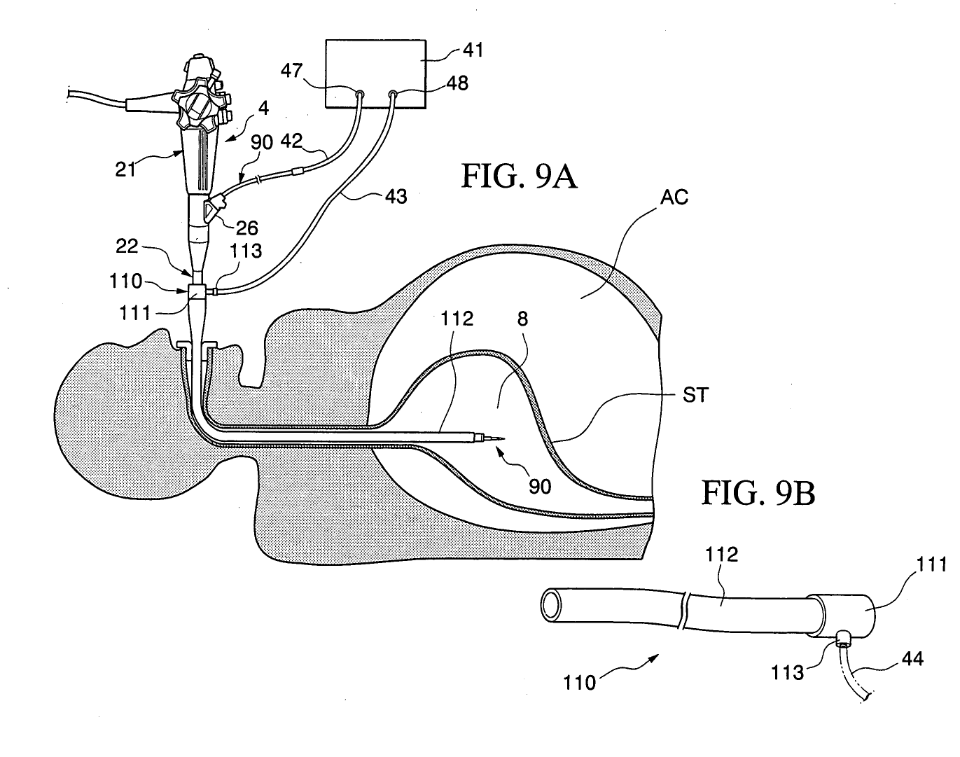

As shown in FIG. 9A, the tube 42 extending from the insufflation metal cap 47 of the air supply device 101 is connected to the lumen of the local injection needle 90. The tube 43 extending from the luminal metal cap 48 is connected to an overtube 110. As shown in FIG. 9 B, a long flexible pipe 112 extends from the proximal end 111 of the overtube 110. An air tight valve (not shown) forming air tight construction between the endoscope 4 and the overtube 110 when the endoscope 4 is inserted into the proximal end 111 is secured as a flange on the inner periphery. A port 113 is provided to which the tube 44 is protrudingly provided further to the distal end than the air tight valve, forming a hole which communicates with the overtube 110.

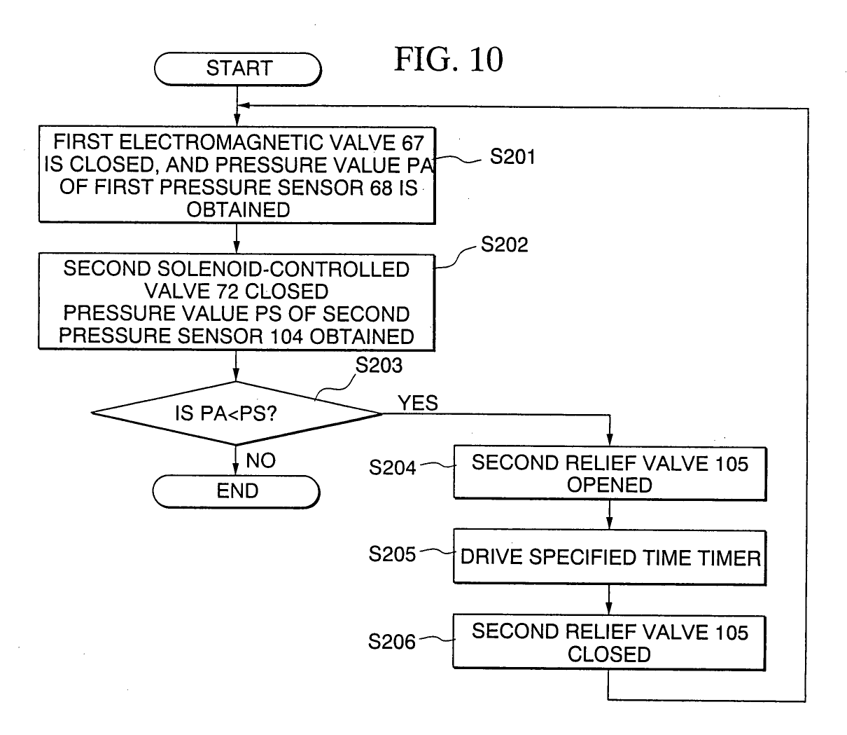

When performing a medical procedure, carbon dioxide gas is supplied to the stomach ST through the inside of the overtube 110, causing inflation, and insulation is performed to the abdominal cavity AC through a local injection needle 90. When cutting the stomach wall, since the pressure value Ps within the stomach ST is higher than the pressure value Pa of the abdominal cavity AC, pressure control is accomplished in accordance with the flow shown in FIG. 10.

Initially, the first solenoid-controlled valve 67 is closed, and the pressure value Pa of the first pressure sensor 68 following the elapse of a predetermined time period is obtained (in step S201). Furthermore, the second solenoid-controlled valve 72 is closed, and the pressure value Ps of the second pressure sensor 104 following the elapse of a predetermined time period is obtained (in step S202). The controller 62 compares the pressure values Pa and Ps, and if the pressure value Ps within the stomach ST is greater than the pressure value Pa of the abdominal cavity AC (Yes in step S203), the second relief valve 105 is opened (in step S204). Since the second relief valve 105 is provided to the second flow path 65 of the air supply device 101, carbon dioxide gas within the stomach ST is discharged outside the body through the air and water supply channel 34. After the second relief valve 105 is opened, the timer of the controller 62 is started, and there is a wait (in step S205) until a predetermined time is reached. After the elapse of a specified amount of time, the second relief valve 105 is closed (in step S206), and the flow returns to step S201. Thereafter, steps S201 to S 202 are repeated until the pressure value Ps within the stomach ST drops to the pressure value Pa of the abdominal cavity AC or less.

The reason the release of the second relief valve 105 occurs only for a specified time is that it is difficult for the second pressure sensor 104 to accurately detect the pressure within the stomach ST while the second relief valve is in the open state since the second relief valve 105 is in the second flow path 65. Also, if the pressure value Ps within the stomach ST is equal to or less than the pressure value Pa of the abdominal cavity AC (in step S203), processing of this flow is terminated. Moreover, if the pressure value Ps within the stomach ST is lower than the pressure value Pa by only a specified value, it would also be acceptable for processing to be terminated.

If the pressure value Ps within the stomach ST is equal to or less than the pressure value Pa within the abdominal cavity AC, the stomach wall is cut, and the endoscope 4 and overtube 110 are advanced, accessing the abdominal cavity AC. Subsequent medical procedures are the same as those of the second embodiment.

In this embodiment, since the pressure sensors 68 and 104 and, and the relief valves 103 and 105 are provided to the path 64 toward the abdominal cavity AC and to the flow path 65 toward the inside of the stomach ST, respectively, independent pressure adjustment can be accomplished. In addition, since the second pressure sensor 104 is attached to the second flow path 65, there is no need to provide a separate tube for pressure measurement, making a piping connection easy. Other effects are the same as those in the second embodiment.

### Fourth Embodiment

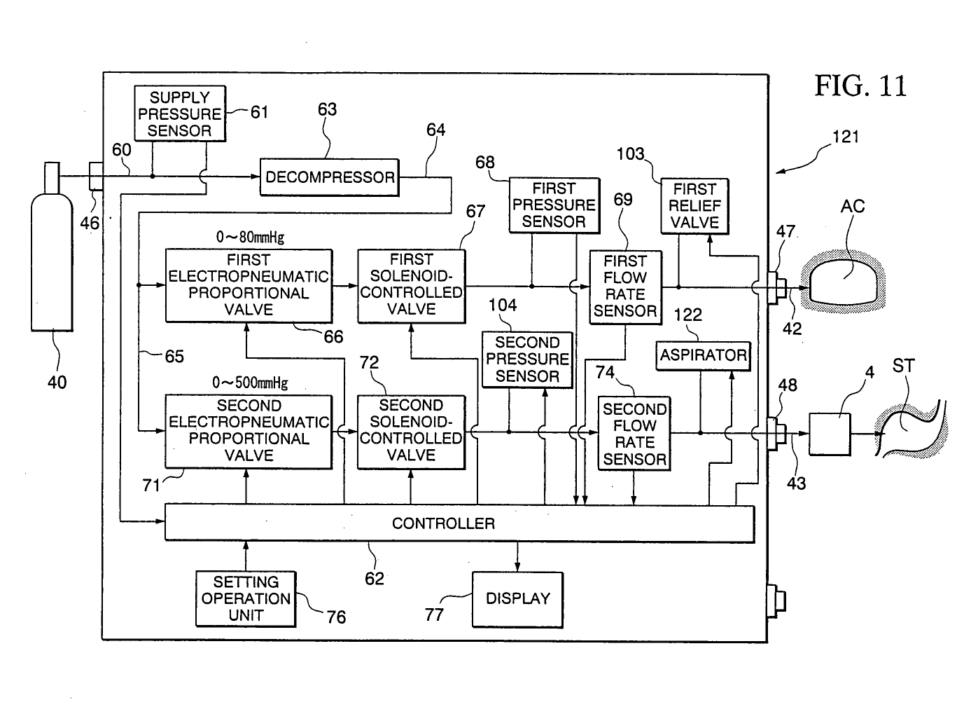

FIG. 11 shows the structure of the air supply device used in the present embodiment

An air supply device 121 is connected to an aspirator 122 in lieu of the second relief valve in the second flow path. The connection of the air supply device 121, the endoscope 4, and overtube 110 is the same as in the third embodiment.

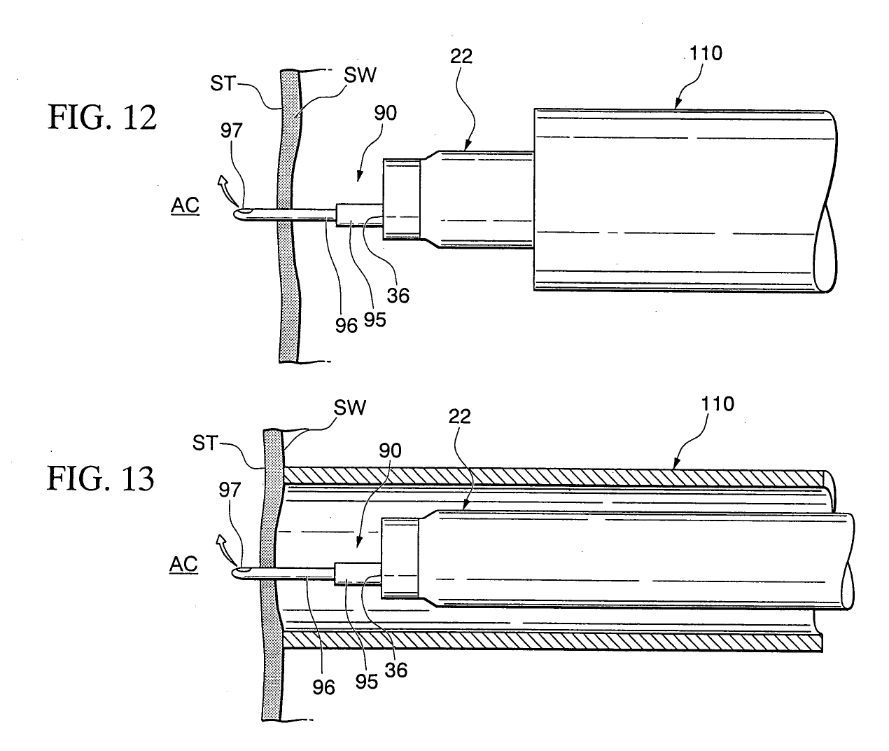

An explanation is provided of the medical procedure and the system of medical treatment. As shown in FIG. 12, after the insertion unit 22 of the endoscope 4 is inserted through the stomach ST, the stomach wall SW is penetrated by the local injection needle 90. Then insufflation is accomplished by supplying carbon dioxide gas to the abdominal cavity AC from the local injection needle 90. Next, carbon dioxide gas is supplied from the overtube 110, inflating the stomach ST until the pressure within the stomach ST is higher than the pressure within the abdominal cavity AC. After confirming the position of an incision, the overtube 110 is advanced while the endoscope 4 is fixed. As shown in FIG. 13, the distal end portion of the overtube 110 is made press against the stomach wall SW, including the position of the incision.

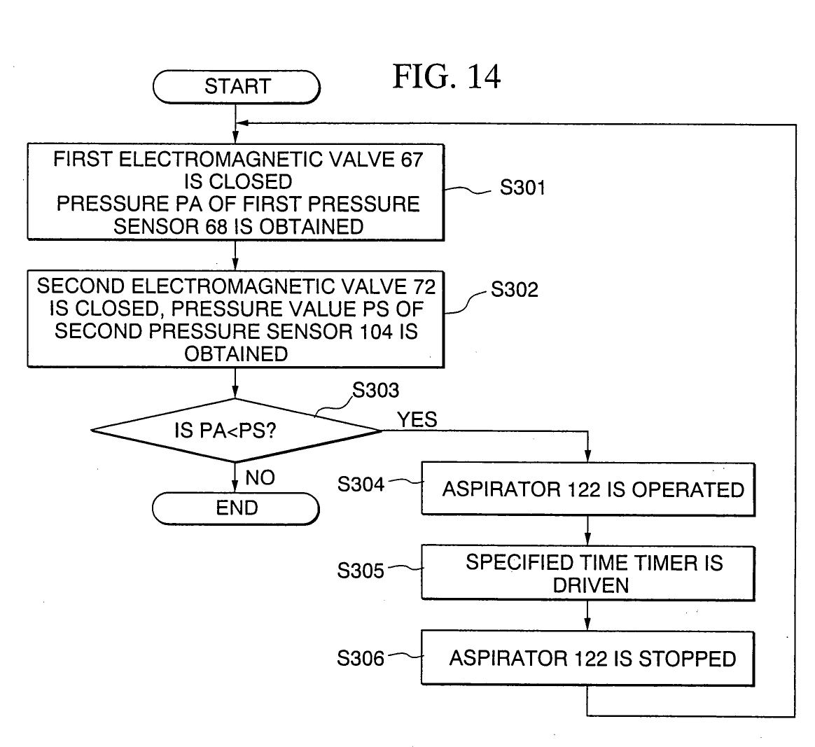

Next, pressure control is accomplished within the stomach ST prior to cutting the stomach wall SW. As shown in the FIG. 14, the first solenoid-controlled valve 67 is closed, and after the elapse of a predetermined time period, the pressure Pa of the first pressure sensor 68 is obtained (in step S301). Further, the second solenoid-controlled valve 72 is closed, and after the lapse of a predetermined time period, the pressure value Ps of the second pressure sensor 104 is obtained (in step S302). If the pressure value Ps is higher than the pressure value Pa (Yes in step S303), the aspirator 122 is operated. Since the second solenoid-controlled valve 72 is closed, carbon dioxide gas within the overtube 110 is aspirated. At the same time, a tinier is started, and there is a wait (in step S304) until a specified amount of time has elapsed, and the operation of the aspirator 122 is stopped (in step S305). Subsequently, the flow returns to step S301. The processes up to this point are repeated, until the pressure value Ps becomes equal to or less than the pressure value Pa (No in step 303), at which point the process is terminated. Moreover, if the pressure value Ps within the stomach ST reaches a value which is lower than the pressure value Pa by only a specified amount, the process may be terminated.

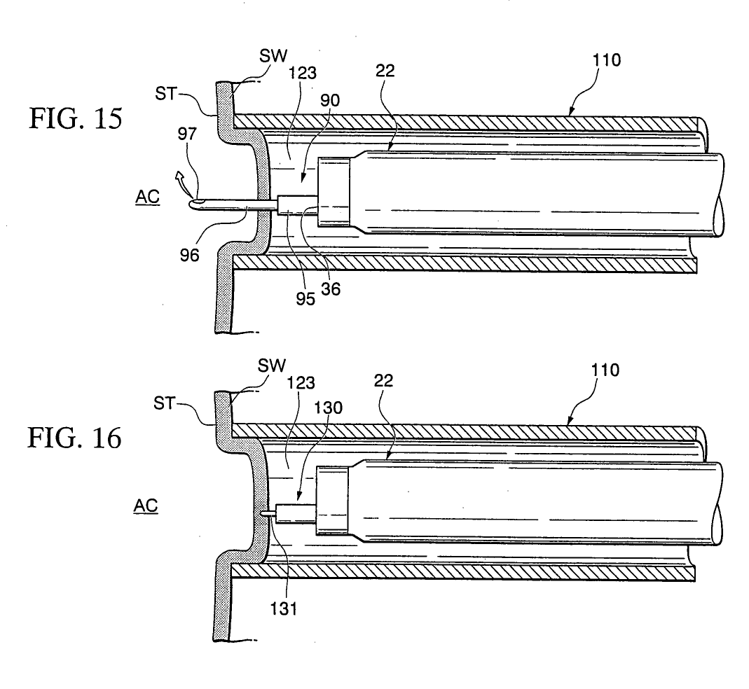

The pressure in the space partitioned by the overtube 110 and the stomach wall SW by means of the aspirator 122 becomes relatively lower than that of the periphery. As a result, as shown in FIG. 15, the stomach wall SW including the target incision position is drawn to the space 123 formed at the distal end of the overtube 110. The local injection needle 90 is removed from the operating channel 36, and is replaced by a high-frequency knife. As shown in FIG. 16, the target incision position is cut by the distal end 131 of the high-frequency knife 130, forming an opening. During this time, since the pressure within the overtube 110 is maintained to be relatively low, even if the opening PO is formed in the stomach wall SW, there is no outflow of fluid from the overtube 110 to the abdominal cavity. The endoscope 4 and the overtube 110 are advanced through the opening PO, accessing the abdominal cavity AC, and the necessary medical procedure is executed. If insufflation is necessary during this period, it is executed from the overtube 110. Upon the completion of a medical procedure, the endoscope 4 is returned to the inside of the overtube 110, i.e., within the stomach ST. Since air supply does not occur within the entire stomach ST, the pressure value Ps within the stomach ST is equal to or less than the pressure value Pa of the abdominal cavity AC, and there is no outflow of fluid from the stomach ST to the abdominal cavity AC.

With this embodiment, in lieu of accomplishing atmospheric release of the entire stomach ST for reducing the pressure value Ps, since aspiration is accomplished by the aspirator 122, the time required to adjust the pressure can be shortened. Furthermore, by accomplishing aspiration with the aspirator 122, since the stomach wall SW including the target incision position is pulled into the overtube 110, a distance can be established between the outside of the stomach wall SW and the other organs or abdominal wall. Owing to this, the stomach wall SW can be opened without being affecting the other organs. Other effects are the same as indicated above.

Moreover, the stomach may be inflated, and marking may be accomplished of the target incision position by means a high-frequency treatment tool or a detainment tool, such as a clip. Furthermore, after marking, and carbon dioxide gas having been supplied in order to inflate the stomach ST may be aspirated with the aspirator 122 to reduce the pressure in the stomach ST. Then, the distal end of the overtube 110 is made press against the stomach wall SW, and the space 123 partitioned by the stomach wall SW, including the overtube 110, may also be aspirated.

### Fifth Embodiment

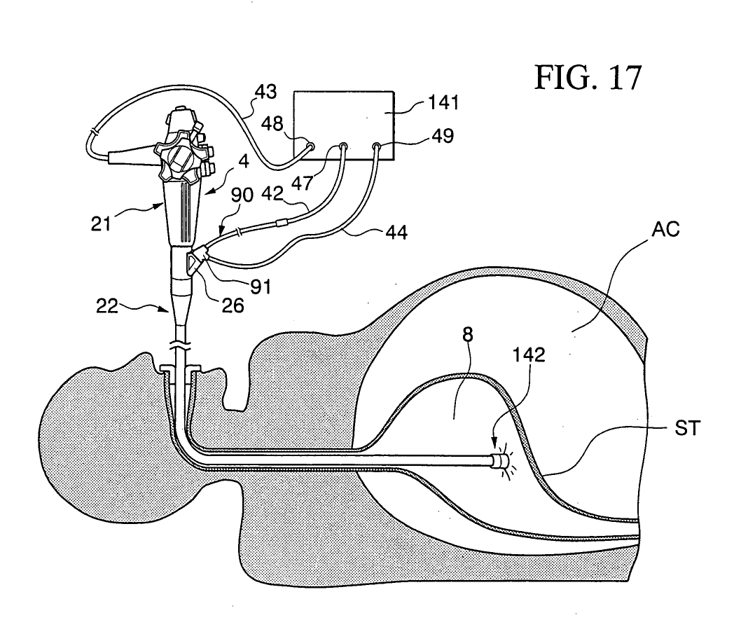

FIG. 17 shows an outline of a system of medical treatment in this embodiment. An insufflation metal cap 47 of an air supply device 141 is connected to the lumen of the local injection needle 90. The metal cap 48 used within the stomach is connected to the air and water supply channel 34 of the endoscope 4. The tube 44 used for pressure measurement within the stomach ST is inserted into the branched port by the stopper 91 of the treatment tool insertion portion 26. Moreover, the air supply device 141, in the air supply device shown in FIG. 6, instead of the second relief valve 73, has a structure in which an aspirator 122 is attached downstream of the second flow rate sensor 74.

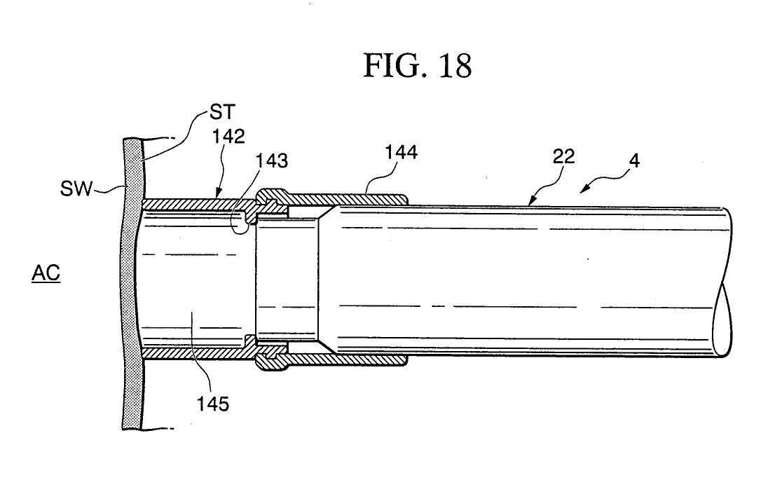

The endoscope 4 is provide with a distal end hood 142 mounted at the distal end of the insertion unit 22. As shown in FIG. 18, the distal end hood 142 has a cylindrical form, and is secured by a ring 144 in a state in which a ring like protrusion 143 within the periphery of the proximal end side presses against the distal end surface of the endoscope.

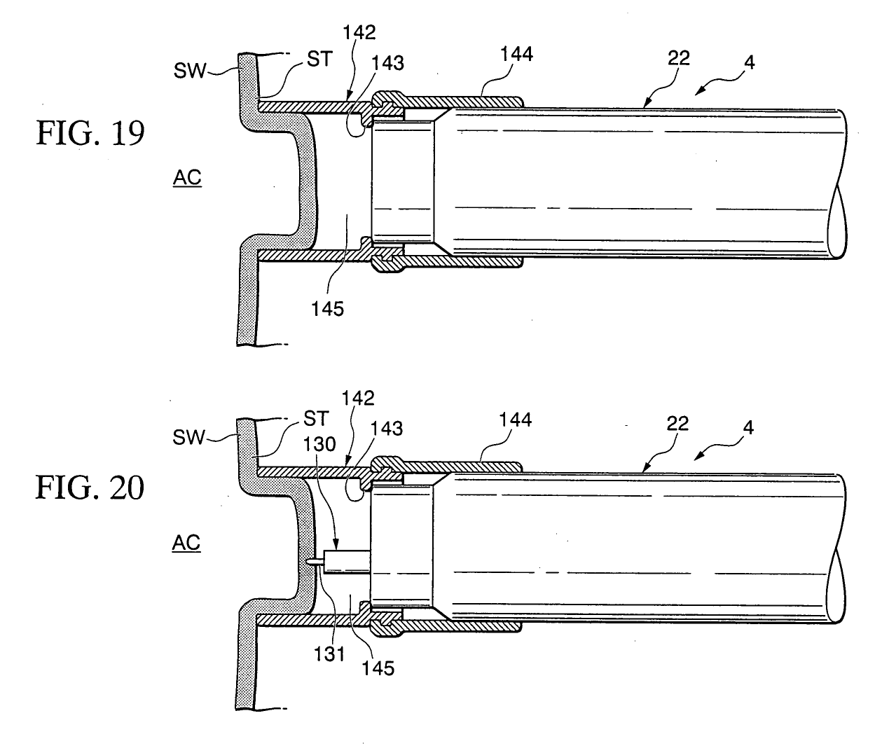

When performing medical procedures, the endoscope 4 is inserted into the stomach ST, and after inflating the stomach ST by supplying air from the air supply device 141, the target incision position is confirmed. After confirming the target incision position, in the same manner as with the fourth embodiment, after pressing the distal end hood 142 against the stomach wall SW including the target incision position, the local injection needle 90 is made penetrate the stomach SW by advancing it within the distal end hood 142. Insufflation is then performed by supplying carbon dioxide gas from the air supply device 141 to the abdominal cavity AC, through the local injection needle 90. Next, the air supply device 141 closes the second solenoid-controlled valve 72, and operates the aspirator 122. As shown in FIG. 19, the fluid from the space 145 formed in the stomach wall SW, and the distal end hood 142 are aspirated through the aspiration channel 34 of the endoscope 4, and the stomach wall SW is pulled into the distal end hood 142 (the pressure of the space 145 enclosed by the distal end hood 142 is lower than the pressure within the abdominal cavity AC). Subsequently, after removing the local injection needle 90, a high-frequency knife is inserted through the operating channel 36.

As shown in FIG. 20, an opening is formed by cutting the target open position with the distal end 131 of the high-frequency knife 130. Since the pressure value Pss within the distal end hood 142 is lower than the pressure value Pa within the abdominal cavity AC, there is no outflow of liquid from the stomach ST side to the abdominal cavity AC. The endoscope 4 is made access the abdominal cavity through the opening SO, and the required medical procedure is executed. Insufflation of the abdominal cavity AC is performed through the channel 34 of the endoscope 4. Upon the completion of the medical procedure, the endoscope 4 is returned to the stomach ST. Since air is not delivered to the entire stomach ST, the pressure value Ps within the stomach ST is equal to or less than the pressure value Pa of the abdominal cavity AC, and there is no outflow of fluid from the stomach ST to the abdominal cavity AC.

According to the present embodiment, the same effects can be obtained as with the fourth embodiment, without the use of an overtube.

An explanation has been provided above of the preferred embodiments of the present invention. However, the present invention is not limited to these embodiments, and additions, abbreviations, and substitutions as well as other exchanges are possible to the extent that they do not departing from the spirit of the present invention. The present invention is not limited to the above explanations, and is restricted only by the scope of the attached claims.

For example, pressure control may be accomplished of the inside of the stomach ST from the delivery of air only to the abdominal cavity AC, without inflating the stomach ST.

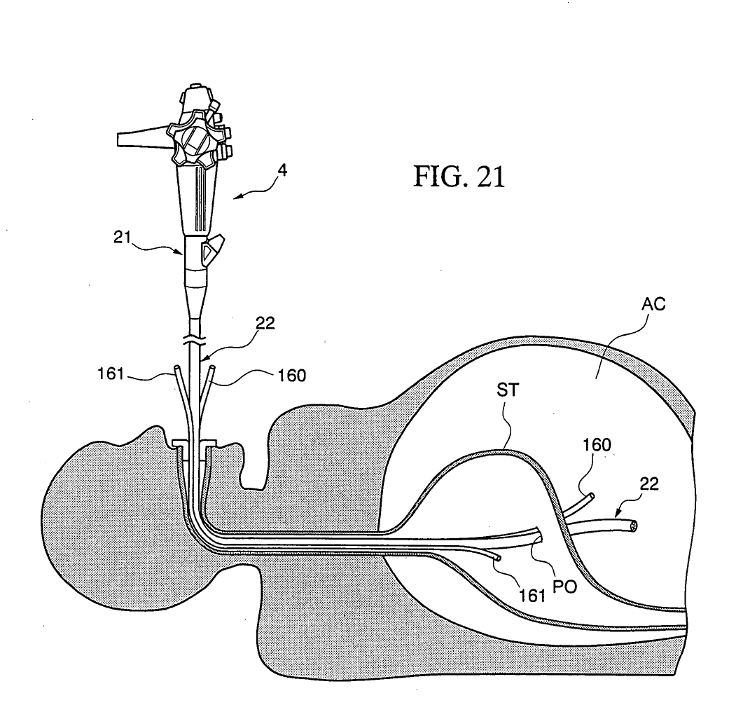

In addition, as shown in FIG. 21, separate from the endoscope 4, a tube 160 which for insufflation within the abdominal cavity AC and an air supply tube 161 for delivering air to the stomach ST may be inserted along the insertion unit 22. The tube 160 is connected to the insufflation metal cap 47 of the air supply device, and has one or two lumens. The tube 160 is introduced to the abdominal cavity AC along with the insertion unit 22, when the endoscope 4 is introduced to the abdominal cavity AC through the stomach wall. When insufflation of the abdominal cavity AC is performed prior to incision, the insufflation needle 50 and the local injection needle 90 are inserted from the operating channel 36, and air is delivered to the abdominal cavity AC. The tube 161 is inserted so that its distal end opening remains within the stomach ST. Even in cases where tubes 160 and 161 are inserted separatedly from the endoscope 4, the same effects are obtained as those indicated above.

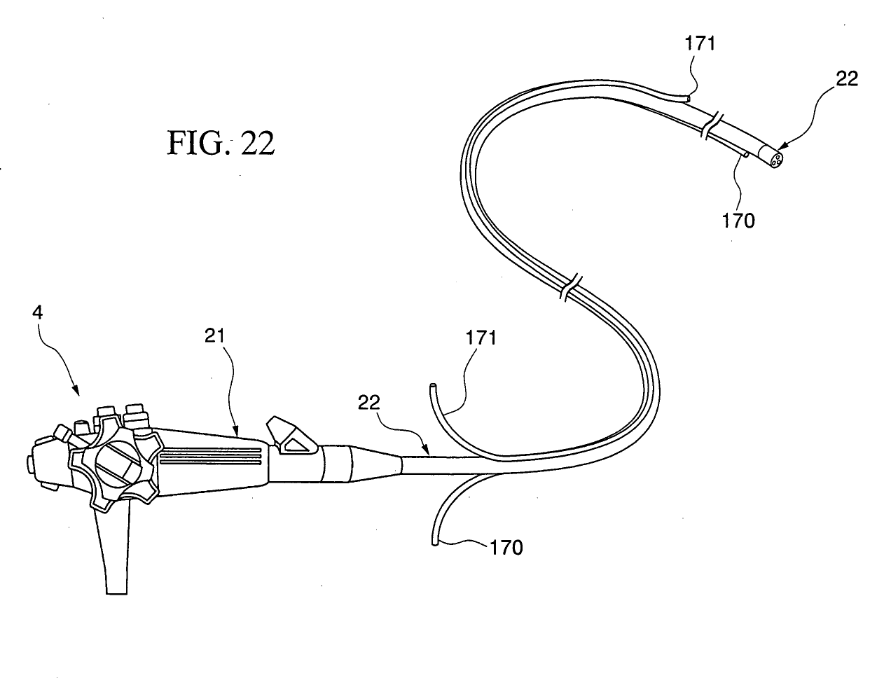

Furthermore, as shown in FIG. 22, tubes 170 and 171 used for air supply may also be fixed to the outer periphery of the insertion unit 22 of the endoscope 4. The tube 170 used for insufflation of the abdominal cavity AC includes one or two lumens, and extend to the distal end of the insertion unit 22. The distal end opening of tube 171 which provides air supply to the stomach ST is arranged more close to the proximal end side than the tube 171. The distal end portion of the tube 171 is capable of being inserted into the stomach ST along with insertion unit 22 of the endoscope 4, and the endoscope 4 may be positioned to remain within the stomach ST even when being introduced through the stomach wall to the abdominal cavity AC. When the endoscope 4 different from tubes 170 and 171 are used, the same effects may be obtained as those indicated above.

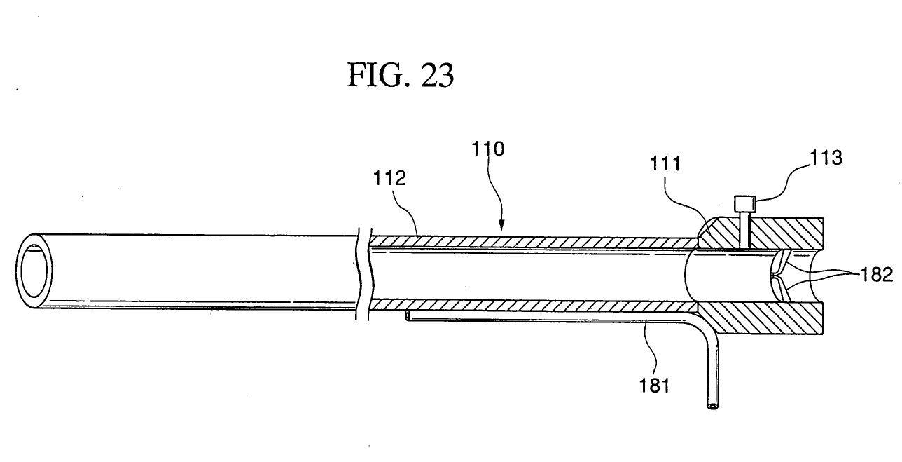

As shown in FIG. 23, with a structure in which the overtube 110 is used as the lumen used for insufflation of the abdominal cavity AC, a tube 181 for air supply to the stomach ST may also be fixed along the outer periphery of the overtube 110. The tube 181 also need not be fixed to the overtube 110. In addition, insufflation may be performed through an attached lumen used for air supply within the overtube 110. Moreover, the port 113 is arranged further to the distal end side from an airtight valve 182. Upon insufflation of the abdominal cavity AC prior to incision, the insufflation needle 50 and the local injection needle 90 are inserted into the abdominal cavity AC and air is supplied through the operating channel 36.

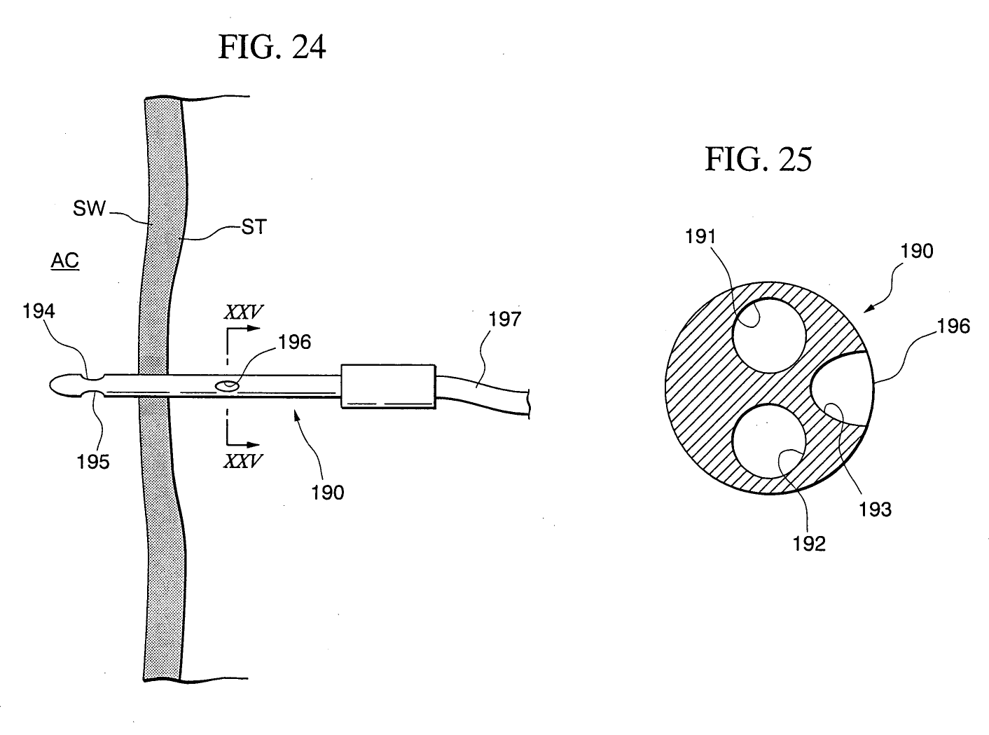

These may also be accomplished of an intragastric insufflation needle 190 such as that shown in FIG. 24 and FIG. 25. The intragastric insulation needle 190 includes three lumens 191, 192 and 193. The insufflation lumen 191 air supply and the lumen 192 used for evacuation of air form openings 194 and 195 the external periphery in the vicinity of the sharp end of the intragastric insufflation needle 190. The third lumen 193 for air delivery and evacuation to and from the stomach forms an opening 196 on the proximal end side, e.g., the outer periphery of the mid-region in the lengthwise direction. The tube 197 includes three independent lumens, to which are respectively attached, lumens 191 to 193 of the intragastric insufflation needle 190, respectively. The insufflation lumen 191 is connected to insulation metal cap 47 of the air supply device. The air evacuation lumen 192 is connected to an aspirator which is not shown. The lumen 193 for air delivery and evacuation to and from the stomach is connected to the metal cap 47 used within the air supply device within the stomach.

The intragastric insulation needle 190, separate from the endoscope 4, is inserted into the stomach ST through the operating channel 36 of the endoscope 4, and is made penetrate the stomach wall SW so that the opening 196 of the lumen 193 remains in the stomach ST. Insufflation of the abdominal cavity AC is performed with the air supplied from the lumen 19 used for abdominal cavity air supply. Next, the stomach ST is inflated through air supply to the stomach ST from the lumen 193 for air delivery and evacuation to and from the stomach. When accomplishing pressure adjustment within the stomach ST, atmospheric release is accomplished of the lumen 193 for air delivery and evacuation to and from the stomach. In addition, smoke produced when using a high-frequency treatment tool in the abdominal cavity AC is discharged from the air evacuation lumen 192 for the stomach of the insulation needle 190. In using the insufflation needle 190 within the stomach, the functions of air supply, air expulsion, and pressure adjustment can be combined into a single needle.

When inserting a pipe path for inflating a hollow organ and a pipe path for insufflation of the abdominal cavity AC from a natural orifice of the body, insertion may be accomplished from different natural orifices. For example, the endoscope may be inserted from the patient's mouth while the tube used for insufflation may be inserted from the anus, or air may be supplied to the abdominal cavity AC through the wall of the large intestine.

The device necessary for performing a specific operation is not limited to the endoscope provided with the observation device and the operating channel referred to in the above embodiment. For example, a device may be attached (hereinafter referred to as a device used for appropriate treatment) which is capable of operating a treatment component from the outside of the body, which includes a treatment component for accomplishing specific treatment at the distal end of the insertion portion inserted into the body. In this case, a medical procedure is accomplished while making observations with an observation device which can be swallowed, such as a capsule endoscope.

What is claimed is:

1. A method for accessing an abdominal cavity, comprising:
introducing a first flow path into the abdominal cavity;
introducing a second flow path into a hollow organ from a natural orifice of the living body;
performing a pressure control so that a pressure within the hollow organ is lower than a pressure of the abdominal cavity by using of the first flow path and the second flow path;
forming an opening in a wall of the hollow organ from the inside of the hollow organ when the pressure within the hollow organ is equal to or less than the pressure within the abdominal cavity, in the step of performing a pressure control; and
inserting a device for performing a medical procedure through the opening.

2. The method for accessing an abdominal cavity according to claim 1, wherein the first flow path is percutaneously introduced into the abdominal cavity.

3. The method for accessing an abdominal cavity according to claim 1, wherein the performing the pressure control comprises:
performing insufflation within the abdominal cavity by using the first flow path; and
releasing gas within the hollow organ by using the second flow path until the pressure within the hollow organ is equal to or less than the pressure within the abdominal cavity.

4. The method for accessing an abdominal cavity according to claim 3, wherein the gas is supplied to the hollow organ prior to the gas within the hollow organ is released.

5. The method for accessing an abdominal cavity according to claim 1, wherein, after being inserted into the hollow organ from the natural orifice of the living body, the first flow path is made penetrate the hollow organ and is introduced into the abdominal cavity.

6. The method for accessing an abdominal cavity according to claim 5, wherein the performing the pressure control comprises:
performing insufflation within the abdominal cavity by using the first flow path; and
releasing gas within the hollow organ by using the second flow path until the pressure within the hollow organ is equal to or less than the pressure within the abdominal cavity.

7. The method for accessing an abdominal cavity according to claim 6, wherein the gas is supplied to the hollow organ prior to the gas within the hollow organ is released

8. The method for accessing an abdominal cavity according to claim 1, wherein the introducing the second flow path comprising inserting an overtube which functions as a device which has an insertion portion inserted into the living body such that a distal end of the second flow path is retained within the hollow organ.

9. The method for accessing an abdominal cavity according to claim 8, wherein the performing the pressure control comprises:
performing insufflation within the abdominal cavity by using the first flow path; and
releasing gas within the hollow organ by using the second flow path until the pressure within the hollow organ is equal to or less than the pressure within the abdominal cavity.

10. The method for accessing an abdominal cavity according to claim 9, wherein the gas is supplied to the hollow organ prior to the gas within the hollow organ is released

11. The method for accessing an abdominal cavity according to claim 1, wherein the introducing the second flow path comprises pressing a cylindrical body communicating with the device against the wall of the hollow organ, and the performing the pressure control comprises aspirating a fluid in the space formed between the cylindrical body and the inner wall of the hollow organ.

12. A accomplished through a natural orifice, comprising:
introducing a first flow path into an abdominal cavity;
introducing the second flow path into the hollow organ from the natural orifice of the living body;
performing a pressure control using the first flow path and the second flow path so that a pressure within the hollow organ is equal to or less than a pressure of the abdominal cavity; and
in a state in which, by performing a pressure control, the pressure within the hollow organ is made equal to or less than the pressure within the abdominal cavity, withdrawing the device which is inserted from the natural orifice of the living body and is introduced into the abdominal cavity through an opening formed in the hollow organ, from the abdominal cavity.

13. The accomplished through a natural orifice according to claim 12, wherein the introducing the first flow path into the abdominal cavity comprises introducing into the abdominal cavity a device having the first flow path, through the opening of the hollow organ.

14. The accomplished through a natural orifice according to claim 12, wherein the introducing the first flow path into the abdominal cavity comprises percutaneously introducing the first flow path into the abdominal cavity.

15. The accomplished through a natural orifice according to claim 12, wherein the introducing the first flow path into the abdominal cavity comprises inserting the first flow path into the hollow organ from the natural orifice of the living body and making penetrate the hollow organ in a position different from the position of the opening.

16. The accomplished through a natural orifice according to claim 12, wherein the introducing the first flow path into the abdominal cavity comprises introducing an overtube which functions as an insertion guide of the device, and the performing the pressure control comprises performing a pressure adjustment in a space between the overtube and the device.

17. The accomplished through a natural orifice according to claim 12, wherein the performing the pressure control comprises:
performing insufflation within the abdominal cavity by using the first flow path; and
releasing gas within the hollow organ by using the second flow path until the pressure within the hollow organ is equal to or less than the pressure within the abdominal cavity.

18. The accomplished through a natural orifice according to claim 12, wherein the gas is supplied to the hollow organ prior to the gas within the hollow organ is released.

19. The accomplished through a natural orifice according to claim 12, wherein, after being inserted into the hollow organ from the natural orifice of the living body, the first flow path is made penetrate the hollow organ and is introduced into the abdominal cavity.

20. The accomplished through a natural orifice according to claim 19, the performing the pressure control comprises:
performing insufflation within the abdominal cavity by using the first flow path; and
releasing gas within the hollow organ by using the second flow path until the pressure within the hollow organ is equal to or less than the pressure within the abdominal cavity.

21. The accomplished through a natural orifice according to claim 12, wherein the introducing the second flow path comprises pressing a cylindrical body communicating with the device against the wall of the hollow organ, and the performing the pressure control comprises aspirating a fluid in the space formed between the cylindrical body and the inner wall of the hollow organ.
